SEQUENCE LISTING (60 sequences)

SEQ ID NO: 1 - protein, 754 aa, Homo sapiens
MIVFLVFKHLFSLRLITMFFLLHFIVLINVKDFALTQGSMITPSCQKGYFPCGNLTKCLPRAFHCDGKDDCGNGADEENCGDTSGWATIFGTVHGNANSVALTQECFLKQYPQCCDCKETELECVNGDLKSVPMISNNVTLLSLKKNKIHSLPDKVFIKYTKLKKIFLQHNCIRHISRKAFFGLCNLQILYLNHNCITTLRPGIFKDLHQLTWLILDDNPITRISQRLFTGLNSLFFLSMVNNYLEALPKQMCAQMPQLNWVDLEGNRIKYLTNSTFLSCDSLTVLFLPRNQIGFVPEKTFSSLKNLGELDLSSNTITELSPHLFKDLKLLQKLNLSSNPLMYLHKNQFESLKQLQSLDLERIEIPNINTRMFQPMKNLSHIYFKNFRYCSYAPHVRICMPLTDGISSFEDLLANNILRIFVWVIAFITCFGNLFVIGMRSFIKAENTTHAMSIKILCCADCLMGVYLFFVGIFDIKYRGQYQKYALLWMESVQCRLMGFLAMLSTEVSVLLLTYLTLEKFLVIVFPFSNIRPGKRQTSVILICIWMAGFLIAVIPFWNKDYFGNFYGKNGVCFPLYYDQTEDIGSKGYSLGIFLGVNLLAFLIIVFSYITMFCSIQKTALQTTEVRNCFGREVAVANRFFFIVFSDAICWIPVFVVKILSLFRVEIPDTMTSWIVIFFLPVNSALNPILYTLTTNFFKDKLKQLLHKHQRKSIFKIKKKSLSTSIVWIEDSSSLKLGVLNKITLGDSIMKPVS

SEQ ID NO: 2 - DNA, 2436 bp, Homo sapiens
gaacttactacatcagaactcctgctgaggtataagaggatacgtctaataactcaattgctgtaaacctatgattgtttttctggtttttaaacatctcttcagcctcagattgattacaatgttctttctacttcatttcatcgttctgatcaatgtcaaagattttgcactgactcaaggtagcatgatcactccttcatgccaaaaaggatattttccctgtgggaatcttaccaagtgcttaccccgagcttttcactgtgatggcaaggatgactgtgggaacggggcggacgaagagaactgtggtgacactagtggatgggcgaccatatttggcacagtgcatggaaatgctaacagcgtggccttaacacaggagtgctttctaaaacagtatccacaatgctgtgactgcaaagaaactgaattggaatgtgtaaatggtgacttaaagtctgtgccgatgatttctaacaatgtgacattactgtctcttaagaaaaacaaaatccacagtcttccagataaagttttcatcaaatacacaaaacttaaaaagatatttcttcagcataattgcattagacacatatccaggaaagcattttttggattatgtaatctgcaaatattgtatctcaaccacaactgcatcacaaccctcagacctggaatattcaaagacttacatcagctaacttggctaattctagatgacaatccaataaccagaatttcacagcgcttgtttacgggattaaattccttgtttttcctgtctatggttaataactacttagaagctcttcccaagcagatgtgtgcccaaatgcctcaactcaactgggtggatttggaaggcaatagaataaagtatctcacaaattctacgtttctgtcgtgcgattcgctcacagtgctgtttctgcctagaaatcaaattggttttgttccagagaagacattttcttcattaaaaaatttaggagaactggatctgtctagcaatacgataacggaactatcacctcacctttttaaagacttgaagcttctacaaaagctgaacctgtcatccaatcctcttatgtatcttcacaagaaccagtttgaaagtcttaaacaacttcagtctctagacctggaaaggatagagattccaaatataaacacacgaatgtttcaacccatgaagaatctttctcacatttatttcaaaaactttcgatactgctcctatgctccccatgtccgaatatgtatgcccttgacggacggcatttcttcatttgaggacctcttggctaacaatatcctcagaatatttgtctgggttatagctttcattacctgctttggaaatctttttgtcattggcatgagatctttcattaaagctgaaaatacaactcacgctatgtccatcaaaatcctttgttgtgctgattgcctgatgggtgtttacttgttctttgttggcattttcgatataaaataccgagggcagtatcagaagtatgccttgctgtggatggagagcgtgcagtgccgcctcatggggttcctggccatgctgtccaccgaagtctctgttctgctactgacctacttgactttggagaagttcctggtcattgtcttccccttcagtaacattcgacctggaaaacggcagacctcagtcatcctcatttgcatctggatggcgggatttttaatagctgtaattccattttggaataaggattattttggaaacttttatgggaaaaatggagtatgtttcccactttattatgaccaaacagaagatattggaagcaaagggtattctcttggaattttcctaggtgtgaacttgctggcttttctcatcattgtgttttcctatattactatgttctgttccattcaaaaaaccgccttgcagaccacagaagtaaggaattgttttggaagagaggtggctgttgcaaatcgtttcttttttatagtgttctctgatgccatctgctggattcctgtatttgtagttaaaatcctttccctcttccgggtggaaataccagacacaatgacttcctggatagtgatttttttccttccagttaacagtgctttgaatccaatcctctatactctcacaaccaacttttttaaggacaagttgaaacagctgctgcacaaacatcagaggaaatcaattttcaaaattaaaaaaaaaagtttatctacatccattgtgtggatagaggactcctcttccctgaaacttggggttttgaacaaaataacacttggagacagtataatgaaaccagtttcctagcaatcattttggatcactggactttcagtggactacctaaaacaggggacagcttttggaagatgacatctgcaatgcttttcatctttaccaacggcaag

SEQ ID NO: 3 - protein, 737 aa, Mus musculus
MWLLLHVILLTEVKDFALADSSMVAPLCPKGYFPCGNLTKCLPRAFHCDGVDDCGNGADEDNCGDTSGWTTIFGTVHGNVNKVTLTQECFLSQYPQHCYCRENELECVKADLKAVPKVSSNVTLLSLKKNKIHRLPVKVFSRYTELRKIYLQHNCITHISRRAFLGLHNLQILYLSHNCITSLRPGIFKDLHQLAWLILDDNPITRISQKSFMGLNSLFFLPMVGNRLEALPETLCAQMPQLNWVDLANNGIKYITNSTFLTCDSLTVLFLPRNQIGFVPEKTFSSLKNLGELDLSSNMITKLPVHLFSDLHLLQKLNLSSNPLLYVHKNQFGSLKQLQSLDLERIEIPNISTGMFQPMKNLSHIYLKTFRYCSYVPHVRICMPSTDGISSSEDLLANGILRVSVWVIAFITCVGNFLVIAVRSLIKAENTTHAMSIKILCCADCLMGVYLFSVGVFDIKYRGQYQKYALLWMESVPCRLLGFLATLSTEVSVLLLTFLTLEKFLVIVFPFSNLRLGKRQTAVALASIWVVGFLIAAVPFTREDYFGNFYGKNGVCFPLHYDQAEDFGSRGYSLGIFLGVNLLAFLVIVISYVTMFCSIHKTALQTAEVRSHIGKEVAVANRFFFIVFSDAICWIPVFVVKILSLLQVEIPGTITSWIVVFFLPVNSALNPILYTLTTSFFKDKLKQLLHKHRRKPIFKVKKKSLSASIVWTDESSLKLGVLSKIALGDSIMKPVSP

SEQ ID NO: 4 - DNA, 2539 bp, Mus musculus
cggactccccagtgacaagcgtcggtgtcccttcgtgccaaggaggtttcagctgagcatgctcagacacatgggaggcaccagactatcaaaactccttctgagaagcaaggacacgtctacgaactcaatgctgcaaatctacagctctttcctctgacaccggacacctctcgagcgcacaccggccgcaatgtggctcctacttcatgtcatccttctgacagaggtcaaagattttgcactggctgacagcagtatggtggctcctctgtgccccaaagggtattttccctgtgggaatctcaccaaatgcttgccccgagcctttcactgcgatggtgtggatgattgcgggaatggtgccgacgaggacaactgtggtgacactagtggatggacaaccatatttggcacagtccatgggaatgtcaataaagtgacattgacacaggagtgctttctcagccagtatccacagcactgttactgcagagaaaatgaactggaatgtgtaaaggctgacttaaaagctgtgccaaaggtttccagcaacgtaacattactatctcttaagaaaaacaaaatccacagacttccagtcaaggtcttcagcagatacacagaactcagaaagatataccttcagcacaactgcatcacacacatctccaggagagcattccttggattacataatctacaaatactgtatctcagccataactgcattacctctctcaggcctgggatattcaaagacttgcatcagcttgcttggctaattttagatgacaacccgatcaccagaatctcacagaagtcctttatggggttaaactccttgtttttcttgcccatggtgggtaaccggctcgaggcccttcctgaaacattgtgtgctcagatgcctcaactcaactgggtggatctggcaaacaatggaataaagtacataacgaactccaccttcctaacgtgcgactcgctcacggttctgtttctgcctagaaatcaaattggttttgttccagagaagacattttcttcattaaaaaatttaggagaactggacctgtctagcaatatgataacaaaactcccagtccaccttttcagcgaccttcatcttctccagaagctgaacctgtcatccaaccctcttctgtatgtccacaagaaccagtttggaagtctcaaacaacttcagtctctagacctggaaaggatagagattccaaacataagcacaggaatgttccagccaatgaagaacctttctcacatttatttgaaaacctttcgatactgctcctatgtcccccatgtccgaatctgtatgccgtcgactgatggtatttcttcgtctgaggacctcttggctaacggtatcctcagagtgtctgtctgggttatagctttcattacctgcgttgggaatttccttgtcatagccgtgagatctctcattaaggctgagaatacaactcacgctatgtccatcaaaatcctttgttgtgccgattgcctgatgggggtgtacctgttctccgtgggcgtctttgacatcaagtaccgagggcagtatcagaagtatgcgctgctgtggatggagagtgtgccctgccgcctgctgggcttcctggccacgctgtccacagaggtctcggtgctgctgctgacattcctgacgctggagaagttccttgtcatagtattccctttcagcaacctgcgcctgggcaagcgccagactgctgtggccctcgccagcatctgggtggtgggatttctcatagcggccgttccgttcaccagagaggattatttcggcaacttttatgggaaaaatggagtctgcttcccacttcattatgaccaagcagaagattttggaagtagagggtactcccttgggattttcctaggtgtgaacttgctggctttcctcgtcatcgtgatttcctatgtcaccatgttctgctccattcataaaacagcccttcagactgcagaagtgaggagccacatcgggaaggaggtggctgttgcaaaccggttcttttttatcgtgttctctgatgccatctgctggatccctgtgtttgtcgttaagatcctgtctctccttcaagtggagataccaggcacaatcacttcctggatcgtggtttttttccttccggtgaacagcgccttaaaccccatcctctacactctgacgacctccttttttaaggacaagttgaaacagttgctgcacaaacatcggaggaaacccatcttcaaagttaagaagaaaagtttatccgcttccattgtgtggacagacgagtcttcacttaaacttggagtgttgagcaaaatagcccttggggacagtataatgaagccggtctccccgtagaggctttggattgctggactttgagggtactacctaaaacaaggtacaatttggggaagatggcatctgagaaggtttccatcttggccagcagccacccactcagcaccagggacacaatggaatggctgt

SEQ ID NO: 5 - DNA, 23 bp, Mus musculus
ctgtccagtgcaccatctggacc

SEQ ID NO: 6 - DNA, 24 bp, Mus musculus
gattacgtaatagtggtccctcag

SEQ ID NO: 7 - DNA, 20 bp, Mus musculus
ctggcttaggtgaagttgct

SEQ ID NO: 8 - DNA, 21 bp, Mus musculus
gaaacagcttatggcgaatga

SEQ ID NO: 9 - DNA, 21 bp, Mus musculus
cctgtcatcggattctttcag

SEQ ID NO: 10 - DNA, 21 bp, Mus musculus
tacaatggccgttgatgacag

SEQ ID NO: 11 - DNA, 21 bp, Mus musculus
tgggaatatggacggtctctt

SEQ ID NO: 12 - DNA, 20 bp, Mus musculus
ccccatgtccgaatctgtat

SEQ ID NO: 13 - DNA, 20 bp, Mus musculus
tcccataaaagttgccgaaa

SEQ ID NO: 14 - DNA, 19 bp, Mus musculus
agaagagcagaatagcagt

SEQ ID NO: 15 - DNA, 18 bp, Mus musculus
accgctcagggtcgaact

SEQ ID NO: 16 - DNA, 18 bp, Mus musculus
catggtgggtaaccggct

SEQ ID NO: 17 - DNA, 19 bp, Mus musculus
gcaatccaaagcctctagc

SEQ ID NO: 18 - DNA, 21 bp, Mus musculus
agcagtatggtggctcctctg

SEQ ID NO: 19 - DNA, 21 bp, Mus muscularis
atggtgggtaaccggctcgag

SEQ ID NO: 20 - DNA, 20 bp, Mus musculus
aaaacagcccttcagactgc

SEQ ID NO: 21 - DNA, 20 bp, Mus musculus
tcccataaaagttgccgaaa

SEQ ID NO: 22 - DNA, 20 bp, Mus musculus
ccacgatccaggaagtgatt

SEQ ID NO: 23 - DNA, 24 bp, Homo sapiens
tcaattgctgtaaacctatgattg

SEQ ID NO: 24 - DNA, 21 bp, Homo sapiens
cttgccgttggtaaagatgaa

SEQ ID NO: 25 - DNA, 21 bp, Homo sapiens
catcagaactcctgctgaggt

SEQ ID NO: 26 - DNA, 21 bp, Homo sapiens
tttttgggaattgcactcatt

SEQ ID NO: 27 - DNA, 22 bp, Homo sapiens
gaaaattaaatgggacaacacg

SEQ ID NO: 28 - DNA, 21 bp, Homo sapiens
cataaagtgaggcacacatgg

SEQ ID NO: 29 - DNA, 25 bp, Homo sapiens
caaattgttaaattttgtcagatgg

SEQ ID NO: 30 - DNA, 21 bp, Homo sapiens
tctgtgcatcacaaatcatgt

SEQ ID NO: 31 - DNA, 22 bp, Homo sapiens
ggctgaatgagttcatttaaca

SEQ ID NO: 32 - DNA, 20 bp, Homo sapiens
gcacaagcaacactattggg

SEQ ID NO: 33 - DNA, 20 bp, Homo sapiens
caagcatgtggcttctaggg

SEQ ID NO: 34 - DNA, 22 bp, Homo sapiens
aaaacatcttacaccaccacga

SEQ ID NO: 35 - DNA, 22 bp, Homo sapiens
ccaacatgagaataggacttcg

SEQ ID NO: 36 - DNA, 21 bp, Homo sapiens
tcgagccttgaaaaagtacca

SEQ ID NO: 37 - DNA, 22 bp, Homo sapiens
cataatcatcatcagtggctca

SEQ ID NO: 38 - DNA, 25 bp, Homo sapiens
tcaaatagtggaaactacattagca

SEQ ID NO: 39 - DNA, 20 bp, Homo sapiens
ggggaggcaggttttatttc

SEQ ID NO: 40 - DNA, 23 bp, Homo sapiens
aagctagtgctagatgtcattgc

SEQ ID NO: 41 - DNA, 21 bp, Homo sapiens
ctccctaatctttgccataca

SEQ ID NO: 42 - DNA, 21 bp, Homo sapiens
tggctagtttccttaatcagg

SEQ ID NO: 43 - DNA, 25 bp, Homo sapiens
ggaggaaagatagtaacaactggaa

SEQ ID NO: 44 - DNA, 20 bp, Homo sapiens
cagtgtcagaccaaggctgt

SEQ ID NO: 45 - DNA, 20 bp, Homo sapiens
actacagcagacgcaaaccc

SEQ ID NO: 46 - DNA, 21 bp, Homo sapiens
ctagtggcaaagcagtctcac

SEQ ID NO: 47 - DNA, 17 bp, Homo sapiens
tggctgactcatacggc

SEQ ID NO: 48 - DNA, 20 bp, Homo sapiens
gattggatgacaggttcctt

SEQ ID NO: 49 - DNA, 19 bp, Homo sapiens
ggaactatcacctcacctt

SEQ ID NO: 50 - DNA, 20 bp, Homo sapiens
gacttcatacatgtcgaatg

SEQ ID NO: 51 - DNA, 22 bp, Homo sapiens
aaaacgtttcatctcaacacca

SEQ ID NO: 52 - DNA, 22 bp, Homo sapiens
ttgttgcatagaaacacattgc

SEQ ID NO: 53 - DNA, 20 bp, Homo sapiens
cccgataggactgcaactgt

SEQ ID NO: 54 - DNA, 21 bp, Homo sapiens
gtgcccaggatgtataattca

SEQ ID NO: 55 - DNA, 21 bp, Homo sapiens
gacattggaaactgatgacat

SEQ ID NO: 56 - DNA, 22 bp, Homo sapiens
cacagtcttgactatgttatct

SEQ ID NO: 57 - DNA, 20 bp, Homo sapiens
gggtaagggatactcaacct

SEQ ID NO: 58 - DNA, 20 bp, Homo sapiens
gtgtcctagactctggcctt

SEQ ID NO: 59 - DNA, 23 bp, Homo sapiens
gcattgactgcaaagtgttattt

SEQ ID NO: 60 - DNA, 20 bp, Homo sapiens
caaaagctgtcccctgtttt

(19) United States
(12) Patent Application Publication
(10) Pub. No.: US 2003/0082650 A1
(43) Pub. Date: May 1, 2003

(54) Great gene and protein

(75) Inventor: Alexander I. Agoulnik, Houston, TX (US)

Correspondence Address: Michael A. Sanzo, Fitch, Even, Tabin & Flannery, Suite 401 L, 1801 K Street, Washington, DC, 20006-1201, US

(73) Assignee: Baylor College of Medicine

(21) Appl. No.: 10/229,735

(22) Filed: Aug. 29, 2002

Related U.S. Application Data
(60) Provisional application No. 60/315,696, filed on Aug. 30, 2001.
(60) Provisional application No. 60/351,432, filed on Jan. 28, 2002.

(51) Int. Cl. G01N 33/567; C07H 21/04; C07K 14/705; C12P 21/02; C12N 5/06

(52) U.S. Cl. 435/7.21; 435/69.1; 435/320.1; 435/325; 530/350; 536/23.5

(57) ABSTRACT

The present invention is directed to a G protein-coupled receptor that is necessary for normal testicular descent during embryonic development. Mutations in this receptor lead to cryptorchidism. In addition to the receptor and genes encoding the receptor, the invention includes assays for mutations in the receptor gene, binding assays which utilize the receptor and transgenic animals which have been engineered to have non-functional receptor alleles. The animals may be used in assays designed to identify agents useful in treating cryptorchidism.

### CROSS REFERENCE TO RELATED APPLICATIONS

The present application claims the benefit of U.S. provisional application No. 60/315,696, filed on Aug. 30, 2001, and No. 60/351,432, filed on Jan. 28, 2002.

### STATEMENT OF GOVERNMENT FUNDING

The United States Government has a paid-up license in this invention and the right in limited circumstances to require the patent owner to license others in reasonable terms as provided for by the terms of NIH Grant No. IR01 HD37067-01A1 awarded by the Department of Health and Human Services.

### FIELD OF THE INVENTION

The present invention relates to a G protein-coupled receptor in mice and humans that promotes testicular descent during male fetal development. Mutations in the gene encoding the receptor result in cryptorchidism. The invention includes binding assays for the receptor and transgenic mice which can be used in assaying test compounds for their effect on cryptorchidism.

### BACKGROUND OF THE INVENTION

Cryptorchidism is a condition in which at least one testes fails to descend fully into the scrotum. It is one of the most frequently observed congenital birth defects with an incidence of 3-4% in newborn boys. Untreated, the condition results in infertility and a significantly increased risk of testicular cancer. Although the causes of cryptorchidism are not fully understood, the Hoxa10 and Insl3 (insulin-like factor 3) genes appear to be required for the normal descent of testes (Rijli, et al., Proc. Nat'l Acid. Sci. USA 92:8185-8189 (1995); Satokata, et al., Nature 374:460-463 (1995); Nef, et al., Nat. Genet. 22:295-299 (1999); Zimmermann, et al., Mol. Endocrinol. 13:681-691 (1999)).

G protein-coupled receptors may also be required for normal testicular descent. Structurally, these membrane associated receptors are characterized by an extracellular N-terminal end, seven hydrophobic alpha helices constituting transmembrane domains and an intracellular C-terminal domain. They bind a wide variety of ligands that trigger intracellular signals through the activation of transducing G proteins (Caron, et al., Rec. Prog. Horm. Res. 48:277-290 (1993); Freedman, et al., Rec. Prog. Horm. Res. 51:319-353 (1996)). Approximately 50-60% of all clinically relevant drugs act by modulating the functions of various G protein-coupled receptors (Gudermann, et al., J. Mol. Med. 73:51-63 (1995)). The identification of specific receptors contributing to cryptorchidism may lead to new methods for the early identification and treatment of this condition.

### SUMMARY OF THE INVENTION

The present invention is based upon the discovery of a new member of the G protein-coupled receptor family which appears to contribute to normal testicular descent during fetal development. It has been given the designation “GREAT” as an acronym for “G protein receptor related to cryptorchidism.” Mutations in the gene for the receptor lead to cryptorchidism in homozygous males and it appears that the receptor binds the Insl3 protein.

In its first aspect, the invention is directed to a substantially pure gene and protein for GREAT derived from either the mouse or human. The term “substantially pure” refers to polypeptides and polynucleotides that have been separated from other accompanying biological components and which should typically comprise at least 85% of a sample, with greater percentages being preferred. Many means are available for assessing the purity of a protein or a nucleic acid, including analysis by polyacrylamide gel electrophoresis, chromatography and analytical centrifugation. In the case of the human, the invention is directed to a protein that consists essentially of the amino acid sequence of SEQ ID NO:1 and to polynucleotides encoding this protein. For the mouse, the protein consists essentially of the amino acid sequence of SEQ ID NO:3. The term “consisting essentially of” or “consists essentially of” refers to amino acid or nucleotide sequences that are identical to those set forth in the sequence identification numbers as well as to other molecules that share at least an 80% sequence identity and which possess the same functional properties with respect to cryptorchidism. Preferred polynucleotides are shown as SEQ ID NO:2 for the human and SEQ ID NO:4 for the mouse.

In addition to the polynucleotides discussed above, the invention includes vectors containing the polynucleotides. Of particular interest are vectors for expressing the human or mouse GREAT protein and having a coding region operably linked to a promoter. The term “operably linked” as used in this context means that the promoter controls the synthesis of the mRNA from the coding region of the vector and a protein having the correct sequence is ultimately produced. The invention also includes host cells that have been transformed with these vectors.

In another embodiment, the invention is directed to an antibody that binds with specificity to either the human or mouse GREAT protein. The term “binds with specificity” as used in this context means that the antibody has at least a 100-fold greater affinity for one of the GREAT proteins than for any other protein normally found in human or mouse cells. The antibody may be made by a process comprising the step of administering the protein encoded by either SEQ ID NO:1 or SEQ ID NO:3 to an animal host at a dosage sufficient to induce antibody formation. The antibodies may be monoclonal or polyclonal.

In addition, the invention is directed to a method of assaying a test compound for its ability to bind to the human GREAT receptor. This involves incubating a source of the human receptor with Insl3 (a ligand for GREAT) and the test compound being examined. The source of the receptor should, preferably, express a large amount of GREAT relative to other receptors and will typically be a cell recombinantly engineered to express the protein. After incubation, the ability of the test compound to bind to the GREAT receptor is determined based upon the extent to which ligand binding has been displaced.

The invention also encompasses assays for determining whether a human subject carries a gene that may lead to cryptorchidism or some other condition related to GREAT expression. In one embodiment, this is done by analyzing the GREAT gene itself to determine whether it carries mutations relative to the sequence of SEQ ID NO:2 and whether these mutations will lead to a protein that differs in sequence from SEQ ID NO:1. The most convenient way for carrying out the assay is using PCR to amplify the region of an individual's genome carrying the GREAT gene and then examining the amplification product for mutations. Alternatively, an assay may be performed by analyzing the GREAT receptor protein to determine whether it has one or more mutations when compared to the amino acid sequence of SEQ ID NO:1.

In another aspect, the invention is directed to a transgenic mouse carrying a mutated gene encoding the GREAT receptor protein of SEQ ID NO:3. Transgenic mice of this type should exhibit crytorchidism when homozygous for the mutation and may be used in assays for determining whether a test compound can be used therapeutically for undescended testes. The method involves interbreeding male transgenic mice that are heterozygous for the mutation with females that are, preferably, homozygous. The test compound may then be administered to pregnant mice resulting from the interbreeding and the extent to which these mice produce homozygous male progeny with undescended testes determined. A comparison can then be made between these results and results obtained for male progeny produced in a similar manner but without the administration of test compound. This procedure may be used to identify new compounds having therapeutic potential.

Assays may also be performed by administering test compounds to mice with undescended testes after birth. A comparison can then be made with similar mice that have not been administered test compound to determine whether testicular descent has been promoted. The objective of such studies is to identify compounds that are highly specific in their action and which can substitute for the relatively non-specific hormonal therapy currently used for neonates.

### DETAILED DESCRIPTION OF THE INVENTION

The present invention is directed to the human and mouse GREAT genes, to vectors containing these genes and to host cells transformed with the vectors. The invention also includes the receptor protein, antibodies against the protein and binding assays in which the protein is expressed and incubated with ligand. In other aspects, the invention encompasses assays for determining whether a particular individual carries a gene predisposing them or their offspring to cryptorchidism, transgenic mice with mutations in their GREAT gene and assays which utilize these mice to test compounds for their effect on cryptorchidism. In the sections below, each of these different aspects of the invention are discussed.

A. Human and Mouse GREAT Genes and Proteins

The invention includes the GREAT receptor protein from humans (SEQ ID NO:1) and mice (SEQ ID NO:3). It also includes genes encoding these proteins (SEQ ID NO:2 and SEQ ID NO:4 respectively). It will be understood that the invention encompasses not only sequences identical to those shown but also sequences that are essentially the same in terms of structure and function. For example, techniques such as site directed mutagenesis may be used to introduce variations into a protein's structure. Variations in the GREAT sequence introduced by this or some similar method are part of the invention provided that the resulting protein retains at least 80% sequence identity and, in addition, retains the same basic biological properties with respect to cryptorchidism.

DNA sequences encoding the proteins of the invention may be obtained from any tissue or cellular source in which they are expressed. Many methods are available for isolating particular DNA sequences and may be adapted in the present case (see e.g., Sambrook, et al., Molecular Cloning: A Laboratory Manual, 2nd ed., Cold Spring Harbor Press (1989)). For example, one method is to screen a cDNA library that has been prepared by reverse transcribing mRNA isolated from tissues or cells that express the gene. The library may be prepared from gonadal or brain tissue and probes (preferably at least 14 nucleotides long) may be synthesized based upon the sequences shown in the Sequence Listing. As an alternative, amplification of the desired sequences may be achieved by the polymerase chain reaction (“PCR”) of reverse transcribed RNA. Primers for PCR may be constructed based upon the sequences shown in SEQ ID NO:2 or SEQ ID NO:4 using guidance provided in the Examples section below.

Expression of recombinant protein may be induced in a host cell by transforming it with an appropriate expression vector. The vector should contain transcriptional and translational signals recognizable by the host, together with the GREAT structural sequence in an operable linkage. For example, the human GREAT DNA sequence should be positioned such that regulatory sequences in the vector control the synthesis of mRNA, and protein having the correct sequence is produced. Preferably, nucleic acids encoding the GREAT receptor are expressed in eukaryotic cells, especially mammalian cells. Such cells are capable of promoting the post-translational modifications necessary to ensure that the recombinant protein is structurally and functionally correct. Examples of appropriate mammalian cells include NIH-3T3 cells, CHO cells, HeLA cells, LM(tk−) cells, and the like. Eukaryotic promoters known to control recombinant DNA expression are preferably utilized to drive the transcription of GREAT DNA. Examples include the mouse metallothionein I gene, the TK promoter of herpes virus, the CMV early promoter and the SV40 early promoter. Transcription may also be directed by prokaryotic promoters such as those capable of recognizing T4 polymerase, the P_{R} and P_{L} promoters of bacteriophage lambda, and the trp, recA, heat shock and lacZ promoters of E. coli.

Expression vectors may be introduced into host cells by methods such as calcium phosphate precipitation, microinjection, electroporation or viral transfer and cells expressing the recombinant protein sequence can be selected by techniques well known in the art. Confirmation of expression may be obtained by PCR amplification of GREAT sequences using primers selected from the sequence shown in SEQ ID NO:2 or SEQ ID NO:4.

Recombinant protein may be purified using standard techniques such as filtration, precipitation, chromatography and electrophoresis. Purity can be assessed by performing electrophoresis on a polyacrylamide gel and visualizing proteins using standard staining methodology. In addition, proteins may be chemically prepared using synthetic methods well known in the art.

B. Antibodies Against GREAT Proteins

The present invention is also directed to antibodies that bind with specificity to GREAT. “Antibodies that bind with specificity” are defined as those that have at least a 100-fold greater affinity for GREAT than for any other similar proteins. The process for producing such antibodies may involve either injecting the GREAT protein itself into an appropriate animal or, alternatively, injecting short peptides made to correspond to different regions of the receptor. The peptides should be at least five amino acids in length and should be selected from regions believed to be unique to GREAT. Thus, highly conserved transmembrane regions, characteristic of G protein-coupled receptors in general, should be avoided in selecting peptides for the generation of antibodies. Methods for making and detecting antibodies are well known to those of skill in the art as evidenced by standard reference works such as: Harlow, et al., Antibodies, A Laboratory Manual, Cold Spring Harbor Laboratory, NY (1988); Klein, Immunology: The Science of Self-Nonself Discrimination (1982); Kennett, et al., Monoclonal Antibodies and Hybridomas: A New Dimension in Biological Analyses (1980); and Campbell, “Monoclonal Antibody Technology” in: Laboratory Techniques in Biochemistry and Molecular Biology (1984).

“Antibody” as used herein includes intact molecules as well as fragments which retain their ability to bind to antigen (e.g., Fab and F(ab′)₂ fragments). These fragments are typically produced by proteolytically cleaving intact antibodies using enzymes such as papain (to produce Fab fragments) or pepsin (to produce F(ab′)₂ fragments). The term “antibody” also refers to both monoclonal antibodies and polyclonal antibodies. Polyclonal antibodies are derived from the sera of animals immunized with antigen. Monoclonal antibodies can be prepared using hybridoma technology (Kohler, et al., Nature 256:495 (1975); Hammerling, et al., In Monoclonal Antibodies and T-Cell Hybridomas, Elsevier, N.Y., pp. 563-681 (1981)). In general, this technology involves immunizing an animal with either intact GREAT or a fragment derived from GREAT. The splenocytes of the immunized animals are extracted and fused with suitable myeloma cells, e.g., Sp₂O cells. After fusion, the resulting hybridoma cells are selectively maintained in HAT medium and then cloned by limiting dilution (Wands, et al., Gastroenterology 80:225-232 (1981)). The cells obtained through such selection are then assayed to identify clones which secrete antibodies capable of binding to GREAT.

The antibodies, or fragments of antibodies, of the present invention may be used to detect the presence of GREAT using a variety of immunoassays. For example, the antibodies may be used in radioimmunoassays or immunometric assays, also known as “two-site” or “sandwich” assays (see Chard, “An Introduction to Radioimmune Assay and Related Techniques,” in: Laboratory Techniques in Biochemistry and Molecular Biology, North Holland Publishing Co., NY (1978)). In a typical immunometric assay, a quantity of unlabeled antibody is bound to a solid support that is insoluble in the fluid being tested. After the initial binding of antigen to immobilize antibody, a quantity of detectably labeled second antibody (which may or may not be the same as the first) is added to permit detection and/or quantitation of bound antigen (see e.g., Radioimmune Assay Method, Kirkham, et al., ed., pp. 199-206, E&S Livingstone, Edinburgh (1970)). Many variations of these types of assays are known in the art and may be employed for the detection of GREAT.

Antibodies to GREAT may also be used in the purification of either intact receptor or fragments of receptor (see generally, Dean, et al., Affinity Chromatography A Practical Approach, IRL Press (1986)). Typically, antibody is immobilized on a chromatographic matrix such as Sepharose 4B. The matrix is then packed into a column and the preparation containing GREAT is passed through under conditions that promote binding, e.g., under conditions of low salt. The column is next washed and bound GREAT is eluted using a buffer that promotes dissociation from antibody, e.g., buffer having an altered pH or salt concentration. The eluted GREAT may be transferred into a buffer of choice, e.g., by dialysis and either stored or used directly.

C. Binding Assays for Human and Mouse GREAT Proteins

One of the main uses for GREAT nucleic acids and recombinant proteins is in assays designed to identify agents capable of binding to the receptor. Such agents may either be agonists, mimicking the normal effect of receptor binding, or antagonists, inhibiting the normal effects of receptor binding. Of particular interest is the identification of agonists that have therapeutic potential in the treatment of cryptorchidism. Based upon a variety of studies, it has been then concluded that the Insl3 protein serves as a natural ligand for GREAT. Insl3 has been previously described and characterized in the art and may be obtained using any known method.

In radioligand binding assays, a source of GREAT is incubated together with a ligand and with the compound being tested for binding activity. The preferred source of GREAT is cells, preferably mammalian cells, transformed to recombinantly express the receptor. The cells selected should not express a substantial amount of any other receptor that might also bind to ligand and distort results. This can easily be determined by performing binding assays on cells derived from the same tissue or cell line as those recombinantly expressing GREAT but which have not undergone transformation.

Assays may be performed either with intact cells or with membranes prepared from the cells (see generally, Wang, et al., Proc. Natl. Acid. Sci. USA 90:10230-10234 (1993)). After incubating the membranes or cells with ligand, and test compound, receptor is separated from the solution containing these components, e.g., by filtration, and the amount of binding that has occurred is determined. Preferably, the ligand is detectably labeled with a radioisotope such as ¹²⁵I . However, if desired, fluorescent or chemiluminescent labels can be used instead. Among the most commonly used fluorescent labeling compounds are fluorescein isothiocynate rhodamine, phycoerythrin, phycocyanin, allophycocyanin, o-phthaldehyde and fluorescamine. Useful chemiluminescent compounds include luminol, isoluminol, theromatic acridinium esters, imidazole, acridinium salts, and oxalate esters. Any of these agents can be used to produce a ligand suitable for use in the assay.

Nonspecific binding may be determined by carrying out the binding reaction in the presence of a large excess of unlabeled ligand. For example, labeled ligand may be incubated with receptor and test compound in the presence of a thousandfold excess of unlabeled ligand. Nonspecific binding should be subtracted from total binding, i.e., binding in the absence of unlabeled ligand, to arrive at the specific binding for each sample tested. Other steps such as washing, stirring, shaking, filtering and the like may be included in the assay as necessary. Typically, wash steps are included after the separation of membrane-bound ligand from ligand remaining in solution and prior to quantitation of the amount of ligand bound, e.g., by counting radioactive isotope. The specific binding obtained in the presence of test compound is compared with that obtained in the presence of labeled ligand alone to determine the extent to which the test compound has displaced receptor binding.

In performing binding assays, care must be taken to avoid artifacts which may make it appear that a test compound is interacting with GREAT when, in fact, binding is being inhibited by some other mechanism. For example, the compound being tested should be in a buffer which does not itself substantially inhibit the binding of ligand to GREAT and should, preferably, be tested at several different concentrations. Preparations of test compound should also be examined for proteolytic activity and it is desirable that antiproteases be included in assays. Finally, it is highly desirable that compounds identified as displacing the binding of ligand to GREAT be reexamined in a concentration range sufficient to perform a Scatchard analysis of the results. This type of analysis is well known in the art and can be used for determining the affinity of a test compound for receptor (see e.g., Ausubel, et al., Current Protocols in Molecular Biology, 11.2.1-11.2.19 (1993)). Computer programs may be used to help in the analysis of results (see e.g., Munson, Methods Enzymol. 92:543-577 (1983); McPherson, Kinetic, EBDA Ligand, Lowry—A Collection of Radioligand Binding Analysis Program, Elsevier-Biosoft, U.K. (1985)).

D. Assays to Determine Whether a Human Carries a Gene Predisposing Cryptorchidism

In a separate aspect, the present invention is directed to an assay for determining whether a person carries a gene that leads to cryptorchidism or some other condition associated with GREAT. Typically, assays are performed on samples obtained from parents prior to the birth of a child, particularly in cases where there is a family history of cryptorchidism. The most direct way to accomplish this is by analyzing the patient's GREAT gene to determine the extent to which it carries mutations relative to the wild-type sequence shown as SEQ ID NO:2. Any mutation which causes a change in the amino acid sequence of the protein would be suggestive of a tendency toward cryptorchidism. Although the invention is not restricted to a particular method of mutational analysis, the most convenient method will generally involve amplifying either the entire GREAT gene or regions of the gene using PCR. The amplified product thus produced may then be analyzed, e.g., by sequencing, to determine the extent to which it carries mutations. Alternatively, assays may be carried out based upon the GREAT protein. In this case, an increased risk of generating cryptorchidism will be evidenced by mutations relative to the sequence shown as SEQ ID NO:1. Analyses may be performed based upon the binding of antibodies to specific regions of the protein or the protein may actually be isolated and sequenced.

E. Transgenic Mice with a Mutated GREAT Gene

Transgenic mice with mutations in the GREAT gene are discussed in detail in the Examples section below. In general, such mice may be produced by: a) preparing a DNA construct comprising a disrupted mouse GREAT gene, in which disruption is accomplished by the insertion of a heterologous marker sequence; b) introducing the DNA construct into a mouse embryonic stem (ES) cell such that the endogenous GREAT gene is disrupted; c) selecting embryonic stem cells comprising the disrupted GREAT allele; d) incorporating the stem cells into a mouse embryo; e) transferring the embryo into a pseudopregnant mouse; f) developing the embryo into a viable offspring; and g) screening offspring to identify heterozygous mice comprising the disrupted gene. Homozygous mice may then be produced by interbreeding the heterozygous mice. The marker genes used in the method should allow for the selection of cells and will typically be an antibiotic resistance gene such as that for neomycin. DNA constructs may also include a second marker for selection such as the HSV-thymidine kinase gene. Various aspects of the method are described in more detail below.

DNA Constructs

The DNA constructs used in the present invention are often referred to in the literature as “knockout” constructs because of their use in disrupting normally active genes. Typically, they contain a relatively long (greater than 1 Kb) targeting segment that has a sequence highly homologous to an endogenous gene in a host cell and that is disrupted by a non-homologous marker sequence. The targeting segment used in constructs may be derived from either genomic or a cDNA by standard methods (see e.g., Sambrook, et al., Molecular Cloning: A Laboratory Manual, Cold Spring Harbor Laboratory Press, Cold Spring Harbor, N.Y. (1989)). For example, a portion of the GREAT gene may be isolated using PCR amplification based upon its known sequence. Alternatively, the targeting segment in a construct may be made using chemical synthesis methods.

In order to incorporate a marker sequence, the targeting segment can be digested with one or more restriction enzymes selected to cut at specific locations. Any location which results in sufficient disruption of the GREAT gene to result in the elimination of a functional gene product after homologous recombination will suffice. Thus, disruption may take place either within the structural sequence of the receptor or at a regulatory element, e.g. the promoter, of the GREAT gene.

The marker sequence used in constructs will typically be an antibiotic resistance gene, e.g. for neomycin resistance, or other gene whose expression can be easily detected and which is not normally present in the host. The marker gene may be expressed in the host cells either as a result of its being operably liked to a promoter in the construct, or by coming under the control of the native GREAT promoter as the result of homologous recombination. In cases where it is part of the construct, the promoter should be selected based upon its having a high activity in the particular host cell undergoing homologous recombination. A typical example of a promoter suitable for use in mouse cells is the promoter of the phosphoglycerate kinase gene. The most preferred gene for use as a marker is a neomycin resistance gene (Neo). Cells which have integrated Neo into their genome and which are expressing this gene are resistant to G418. Thus, a simple means is provided for selecting recombinant cells. In addition to a promoter, the marker gene will typically have a polyA sequence attached to its 3′ end.

In addition to a marker gene used for disrupting the GREAT receptor and for identifying cells that have undergone homologous recombination, the constructs of the present invention will typically include a gene that can be used for distinguishing between cells in which recombination has occurred at the GREAT locus and cells in which recombination has occurred elsewhere in the genome. Preferably, this “selection sequence” will consist of the HSV-thymidine kinase gene under the control of an appropriate promoter. The combination of a marker sequence for selecting all cells that have undergone homologous recombination and a selection sequence for distinguishing site specific integration from random integration has been termed “positive-negative selection” and details of both the procedure and the production of constructs appropriate for the procedure are well known in the art (see, Capecchi, TIG 5:70 (1989); Mansour, et al., Nature 336:348 (1988), Thomas, et al., Cell 51:503 (1987) and Doetschman, et al., Nature 330:576 (1987)).

The DNA construct for disruption of the GREAT gene may be transfected directly into host cells or it may be first placed in a vector for amplification prior to transfection. Preferred vectors are those that are rapidly amplified in bacterial cells such as the pBluescript IISK vector (Stratagene, San Diego, Calif.) or pGEM 7 (Promega Corp., Madison, Wis). DNA constructs may be either circular or linear. However, it is generally preferred that prior to transfection into host cells, circular constructs be linearized.

Production of Host Cells Comprising DNA Constructs

The constructs described above must next be transferred into embryonic stem (ES) cells. Any ES cell line capable of integrating into and becoming part of the germ line of a developing embryo may be used, e.g. the murine cell line D3 (ATCC, 12301 Parklawn Drive, Rockville, Md., CRL 1934). After appropriate host cells have been chosen, they are cultured and prepared for DNA insertion using methods well known in the art (see e.g., Robertson in Teratocarcinomas and Embryonic Stem Cells: A Practical Approach, IRL Press, Washington, D.C. (1987); Bradley, et al., Current Topics in Devel. Biol. 20:357-371 (1986); and Hogan, et al., Manipulating the Mouse Embryo: A Laboratory Manual, Cold Spring Harbor Laboratory Press, Cold Spring Harbor, N.Y. (1986)).

The introduction of construct into host cells can be accomplished using any of a variety of means including electroporation, microinjection or calcium phosphate treatment. The screening of transfected cells may be also carried out using several different methods. In cases where an antibiotic resistance gene has been used as a marker, cells can be cultured in the presence of antiobiotics to identify recombinants. In cases where other types of markers are used, Southern hybridizations may be carried out using labeled probes specific for the marker sequence. Finally, if the marker gene encodes an enzyme whose activity can be detected (e.g., beta-galactosidase), enzymatic assays may be performed.

It will usually be desirable not only to identify cells in which recombination has occurred, but also to distinguish specific recombination, i.e. integration at the GREAT gene locus, from random insertion events occurring elsewhere in the genome. To identify cells with proper integration, chromosomal DNA can be extracted from cells using standard methods and Southern Hybridizations can be performed using probes designed to bind specifically to DNA derived from constructs. Alternatively, PCR amplification can be performed using primers that will only act in cells where homologous recombination has occurred at the receptor locus or which will produce a distinctive product of known size from such cells.

One way to enrich preparations for recombinants modified at the GREAT locus is to incorporate the HSV-thymidine kinase gene into constructs at a position adjacent to the targeting segment. The construct is designed so that the HSV-tk gene is only transferred to the host cell genome when recombination occurs at the GREAT gene site. Because the HSV-tk gene makes cells susceptible to the drug gangcylovir, the exposure of recombinants to this drug will negatively select against cells in which random integration has occurred. See Mansour, et al., Nature 336:348 (1988)).

It will be appreciated that homologous recombination will result in the disruption of one GREAT allele much more frequently than in the disruption of both alleles. If one desires to produce cells that are completely deficient in the GREAT receptor, it may therefore be necessary to conduct a second round of homologous recombination on cells that have already been selected as having one allele disrupted. In the second round of transfection, a marker should be used that is different from the marker used in producing the initial recombinants. For example, if a neomycin resistance gene was used to produce cells with one disrupted allele, beta-galactosidase may be used as a marker in the second construct. Screening for cells that have incorporated DNA at the GREAT site may be carried out as described above.

Development of Transgenic Animals

Embryonic stem cells engineered to contain a mutant GREAT allele and produced by homologous recombination as described above, may be used to make transgenic animals with a substantial absence of functional GREAT. The first step in this process is to incorporate the mutant ES cells described above into an embryo. The preferred method for accomplishing this is by microinjection into an embryo at the blastocyst stage of development. In mice, blastocysts at about 3.5 days of development may be obtained by perfusing the uterus of pregnant animals (see Bradley, in Treatocarcinomas and Embryonic Stem Cells: A Practical Approach, Robertson, ed., IRLP Press, Washington, D.C. (1987)). Preferred blastocysts have genes coding for a phenotypic marker that is different from the corresponding genes in the stem cells. In this way, offspring are produced that can easily be screened for the presence of mutant GREAT receptor alleles. For example, if the ES cell line carries the gene for a white coat, the embryo selected will, preferably, carry the gene for a black or brown coat and offspring carrying a mutant GREAT allele should have mosaic coats.

After the ES cells have been incorporated into the blastocysts, the chimeric embryo is implanted into the uterus of a pseudopregnant animal. Such animals may be prepared by mating females with vasectomized males of the same species. The pseudopregnant stage of the female is important for a successful implantation and will vary from species to species. For mice, females about two or three days pseudopregnant should typically be used.

After chimeric embryos have been implanted into pseudopregnant animals, they are allowed to develop to term and offspring are then screened for the presence of a mutant GREAT allele. In cases where a phenotype selection strategy has been employed, e.g., based upon coat color as described above, initial screening may be accomplished by simple inspection of animals for mosaic coat color or some other readily apparent phenotypic marker. In addition, or as an alternative, chromosomal DNA may be obtained from the tissue of offspring, e.g. from the tail tissue of mice, and screened for the presence of a modified nucleotide sequence at the GREAT gene locus using Southern blots and/or PCR amplification.

Once offspring have been identified carrying the GREAT gene mutation, they can be interbred to produce homozygous animals characterized, in the case of males, by cryptorchidism. Heterozygotes may be identified using Southern blots or PCR. Other means for identifying and characterizing transgenic animals are also available. For example, Northern blots can be used to probe mRNA obtained from tissues of offspring for the presence or absence of transcripts encoding either the GREAT receptor, the marker gene, or both. In addition, Western Blots may be used to assess GREAT expression by probing with antibody specific for the receptor.

F. Assays Utilizing Transgenic Animals

The transgenic animals which have been engineered to have defective GREAT genes may be used in assays designed to identify drugs useful in the treatment of cryptorchidism (or other conditions associated with abnormal GREAT expression). This is based upon the finding that male mice homozygous for mutations in GREAT invariably have undescended testes but that heterozygous males and homozygous females are fertile. Thus, heterozygous males may be interbred with homozygous females to produce offspring, a portion of which will be homozygous for mutations and, in the absence of any other factor, will exhibit cryptorchidism. Test compounds may be administered to mice by injection at several different concentrations and at several different times during gestation. Homozygous males are easily identified due to the fact that they contain white spots on their coat. Animals showing such spots with normal testicular development would be an indication that a test compound was having a positive effect in preventing cryptorchidism.

Alternatively, test compounds may be given to neonatal mice with undescended testes. Compounds may be administered at several doses and the results obtained in promoting testicular descent compared with the results obtained from similar mice who do not receive test compound. It is entirely plausible that a compound may be effective at this time since testicular descent in mice is not usually completed until about 20 days after birth. A similar situation exists in humans in whom testicular descent is finalized during the first year of life. At present, conventional therapy involves LH-RH injections after 6 months of age, followed by HCG administration for non-responders, orchidopexy (surgery) for non-responders and later, LH-RH analogues for patients with low germ cell count or no spermatogonia (Hadziselimovich, Horm. Res. 29:55 (2001)). The ultimate objective of assays is to identify compounds that are highly specific in promoting the descent of testes and that can be used without the side effects associated with hormone therapy.

### EXAMPLES

### Example 1

### First Mouse Study

Below, a novel mouse insertional mutation is described that causes cryptorchidism with white spotting (crsp). About half of the homozygous mice of both sexes die within first 24 hours after birth. Surviving males homozygous for this mutation exhibit high intraabdominal cryptorchidism, which is associated with complete sterility. Homozygous females are fertile. Surgically descended testes in crsp/crsp males show normal spermatogenesis, indicating that the genetic defect in these mice is specific for testicular descent rather than germ cell differentiation. Crsp has been mapped to mouse chromosome 5, proximal to the Brca2 gene, a region homologous to human chromosome 13q12-13. The transgenic insertion was accompanied by a deletion at the site of integration. We have assembled a BAC contig spanning the critical region and identified a candidate gene that is deleted as a result of the transgenic insertion.

I. Materials and Methods

Production of the Transgenic Mice

A 3-kb tyrosinase minigene construct (Trp2-tyro) containing a mouse tyrosinase CDNA under the control of a tyrosinase-related protein 2 (Trp2) promoter was used for standard pronuclear microinjection of albino FVB/N embryos. The male transgenic founder mouse for the crsp family (OVE 754) had an agouti fur color and was crossed to FVB females to establish the mutant transgenic line. The progeny were analyzed for inheritance of the transgene by coat pigmentation and PCR using primers specific for the transgenic construct (TyEx1, 5′-CTGTCCAGTGCACCATCTGGACC-3′ (SEQ ID NO:5) and TyEx2, 5′-GATTACGTAATAGTGGTCCCTCAG-3′ (SEQ ID NO:6)). Phenotypic analysis showed that the transgenic insertion and coat color phenotype cosegregated in more than 1,000 transgenic mice.

Cloning the Transgene Insertion Breakpoints

A phage library was constructed from crsp/crsp genomic DNA using a partial digest with Mbol and ligation into the EMBL3 (Promega) vector. The library was screened with the tyrosinase CDNA. Plaques containing the transgenic minigene were distinguished from clones of the endogenous tyrosinase gene by inter-exonic PCR using primers TyEx1/TyEx2. The ends of the genomic inserts were sequenced using vector-specific primers and the sequences were used to design genomic STS primers. To verify the chromosomal origin of the recovered sequences, we tested for amplification of DNA from a mouse chromosome 5/hamster hybrid cell line.

Meiotic Mapping of crsp

DNA from a Jackson Laboratory interspecific backcross panel BSS was typed for crsp using primers 2TYF (5′-CTGGCTTAGGTGAAGTTGCT-3′, SEQ ID NO:7) and 2TY25R (5′-GAAACAGCTTATGGCGAATGA-3′, SEQ ID NO:8) located 5 kb proximal to the crsp transgene insertion site. The primers amplify an 800-bp product from C57BL/6 (B6) DNA and 1.2-kb fragment from Mus spretus. A total of 94 mice were genotyped.

Fluorescent In Situ Hybridization

Spleen lymphocytes from a hemizygous transgenic male mouse were cultured and metaphase chromosome spreads prepared. The tyrosinase CDNA was labeled with digoxigenin (Bocringer Mannheim) by nick translation. A solution containing 200 ng labeled DNA, 10 μg salmon sperm DNA and 5 μg mouse Cot-1 DNA (Gibco BRL) was used to probe previously G-banded slides. Slides were hybridized overnight and unbound probe was removed by washing in 1×SSC at 72° C. Probe DNA was detected with anti-digoxigenin FITC (Boehringer Mannheim) and chromosomes were counterstained with 0.2 μg/ml propidium iodide in an antifade solution. Images were captured using a PowerGene probe analysis system (Perceptive Scientific Instruments, League City, Tex.).

Histological Analysis

Testes were fixed in Bouin's solution, embedded in paraffin, sectioned, and stained with hematoxylin-eosin according to standard protocols. Photomicrographs were taken with an Axiolab microscope equipped with an automatic camera (Karl Zeiss).

RACE- and RT-PCR

RACE (rapid amplification of cDNA ends) PCR was done to identify the 5′ end of the mouse cg003 gene using a Marathon testis cDNA kit (Clontech, Palo Alto, Calif.). A gene specific antisense primer cg003MR (5′-CCTGTCATCGGATTCTTTCAG-3′, SEQ ID NO:9) designed from the mouse cg003 EST (AI606639) and nonspecific adapter-primers from the Marathon cDNA kit were used in the PCR. The resultant fragment was cloned into the pCR vector (Invitrogen) and sequenced (GenBank AF346502). Primers cg003-13F (5′-TACAATGGCCGTTGATGACAG-3′, SEQ ID NO:10) and cg003-14F (5′-TGGGAATATGGACGGTCTCTT-3′, SEQ ID NO:11) amplify a 187-bp fragment from the first exon of the cg003 gene, which is deleted in Crsp mice. Great specific primers Rec2F (5′-CCCCATGTCCGAATCTG TAT-3′, SEQ ID NO:12) and Rec4R (5′-TCCCATAAAAGTTGCCGAAA-3′, SEQ ID NO:13) were used to amplify a 526-bp cDNA fragment. Complete cDNA sequence and intron/exon organization of the Great gene (GenBank AF346501) was established by RACE-PCR, testis cDNA library screening, and comparison of the cDNA and genomic sequences.

For RT-PCR, total RNA was extracted from mouse organs with TRIzol reagent (GIBCO BRL). RNA was then treated twice with RNAse-free DNAse (Promega) to remove contaminating DNA. After first strand synthesis with SuperScript II reverse transcriptase (GIBCO BRL), aliquots were subjected to PCR analysis.

Surgical Descent of the Cryptorchid Testis (Orchiopexy)

After induction of peritoneal anesthesia in 4-week-old crsp/crsp males, the abdominal cavity was opened with a small midline vertical incision. The right testis was brought out of the abdominal cavity and steadied in a superficial skin pouch in the region of the scrotum by suturing its capsule to the skin. The left testis was left untreated. Mice were sacrificed after 60 days. Both testes were removed and processed for histology.

II. Results

Cryptorchidism in crsp/crsp Mice

The crsp mutation was identified in a transgenic family (OVE 754) carrying a mouse tyrosinase minigene. Mice heterozygous for the transgene were intercrossed to produce homozygous animals. Among the F2 generation, several mice had a white spot on their back and/or abdomen. White-spotted females were fertile, and 100% of their offspring were transgenic and pigmented, implying that the white spotting is associated with homozygosity for the transgene. All white-spotted males are infertile. Internal examination of the adult white-spotted homozygous males showed that the mice have small, bilateral undescended testes located in a high intraabdominal position. The scrotal sac is not fully formed. Vas deferens, epididymis, and accessory glands are present. Males are normally virulized, seminal vesicles are of a normal size and peripheral blood testosterone levels are within the normal range. Both testes in the mutants are located beneath the peritoneum near the kidneys.

There is a clear reduction in the size of the testes and epididymis in mutant mice. Histological evaluation of the testis from adult crsp/crsp males reveals complete arrest of spermatogenesis. In the seminiferous tubules from heterozygous males, all stages of spermatogenesis, including mature sperm, can be seen whereas in the adult mutants only a few spermatogonial cells are present in the cryptorchid testis. Vacuolization and lesions of the seminiferous epithelium are found in mutants. These defects are typical of cryptorchid testes described in humans and other animals and explain the infertility of Crsp males. Differences in the genital track between homozygous mutants and wild-type males are clearly visible at day 1 after birth. The position of the testes in the mutants is high, near the kidney. The mutant mice have an extended gubernaculum and fail to form the inguinal canal. Nevertheless, at birth, mutant and normal testes are histologically indistinguishable. At day 15 after birth, the cryptorchid testes are about 50% smaller than normal, but histologically the testes are still fairly normal.

Crsp Does Not Affect Spermatogenesis Directly

To determine whether spermatogenesis can be rescued in the mutant males, we performed orchiopexy. The right testes of 4-week-old mutant males were removed from the abdominal cavity and placed under the skin in the scrotal area. The left testes were left untreated. Sixty days after surgery, there was a significant difference between the untreated and the experimentally descended testes. Spermatogenesis was restored in the relocated testes, with all stages of germ cell differentiation present including mature spermatozoa. Mature sperm were observed even in the epididymis of one of the operated males. Thus, the ersp mutation does not alter the development of the male germ cells directly. Sterility of the cryptorchid males reflects the high temperature environment of the intraabdominal testes.

White Spotting, Neonatal Lethality, and Decreased Body Weight

Mice homozygous for the crsp mutation exhibit white spotting that becomes visible at 3-5 days after birth with the development of skin pigmentation. The mice show a variable sized white streak on the back in the midtrunk region plus a white belly patch. The two spots may coalesce to form a white belt.

In matings between crsp/+ heterozygotes or crosses of heterozygous males to homozygous females, some of the newborn mice die within the first 24 hours after birth. Examination of the breeding records indicated that at 2 weeks after birth the number of homozygous mutants was about 50% of the expected number. An analysis of the genotype of 30 dead newborns revealed that all of them were crsp/crsp. No gross anatomical abnormalities were found in these animals. Histological examination did not reveal any obvious differences between mutant and wild-type mice in their main organ systems.

Homozygous crsp/crsp newborns were found to have 15% to 25% decreased body weight in comparison with their heterozygous crsp/+ or wild-type +/+ littermates. The mutant animals continue to manifest a clear weight reduction during the first 3 months after birth, but by 4 months of age homozygotes reach the weight of their normal littermates.

Transgenic Insert Maps to Chromosome 5

Southern blot hybridizations of genomic DNA from transgenic and wild-type animals indicated that about 8-10 copies of the transgene had integrated. To localize the insertion site cytogenetically, a FISH analysis was performed using the tyrosinase minigene as a probe. Only one hybridization signal was detected, located on the distal part of chromosome 5, indicating a single integration site of the transgene. No rearrangements of chromosome 5 were detected at the level of G-banding of metaphase chromosomes. Analysis of meiotic pairing in heterozygous males did not reveal any abnormalities.

In order to obtain genomic probes corresponding to the transgenic integration site, we cloned junction fragments containing transgenic and adjacent mouse genomic DNA. These fragments were isolated from a crsp/crsp EMBL3 lambda genomic library using tyrosinase cDNA as a probe. Ends of the genomic inserts from positive phages were sequenced and compared with the tyrosinase CDNA to identify flanking genomic sequences.

PCR analysis with primer pairs derived from one of the identified breakpoints revealed a size polymorphism between bands amplified from C57BL/6 and Mus spretus mouse genomic DNAs. We mapped this polymorphic marker on a Jackson Laboratory Interspecific Backcross Panel. The polymorphism maps to the distal part of chromosome 5 and shows no recombination with the mouse Brca2 gene located at 84 cM. This part of mouse chromosome 5 is syntenic to human chromosome 13q12-13, which has been well characterized previously (Couch et al., Genomics 36:86-99 (1996)).

Physical Mapping of the crsp Interval

Two sets of markers were used to assemble a physical map of the crsp region. First, mouse homologues of human genes previously discovered in the vicinity of BRCA2 were identified. Human cDNA sequences were used for BLAST homology searches of the mouse Expressed Sequence Tag (EST) database. A series of PCR primers was designed based on the identified mouse exons. As a second set of markers STSs from the physical map of mouse chromosome 5 were used. Assays of the YAC contig WC5.54, which contains the mouse Brca2 gene allowed us to determine the relative position of the markers on the contig. The crsp integration site was found to map proximal to Brca2, at the 5′ end of the cg003 gene.

We subsequently analyzed crsp/crsp genomic DNA by PCR using STSs that map proximal to the transgenic integration site. There was no amplification with markers M1983, M10602, D5Mit122, and D5MIT286.2, indicating that a genomic deletion occurred during integration of the transgene. The genetic positions of the deleted marker D5Mit192 (81cM) and one nondeleted marker D5Mit192 (82cM) on the integrated MIT SSLP/Copeland-Jenkins RFLP genetic map indicate that the deletion in the crsp mutant lies proximal to Brca2.

Using STS markers from the deleted region, a BAC contig has been constructed that spans the entire crsp interval. The SP6 end of BAC clone 337K7 is not deleted in the crsp/crsp mice, defining the maximum extent of the deletion. We have estimated that the total length of the deletion is less than 550 kb.

Genes Deleted in crsp

The mouse homologue of the human gene CG003 is located in the immediate proximity of the transgenic insertion. A BLAST search of the mouse EST databank showed several sequences homologous to the middle part of the human cDNA. Using RACE PCR on testis RNA, an alternative first 5′-exon of the mouse cg003 CDNA (GenBank accession number AF346502) was identified. In human genomic DNA homologues this exon is located about 85 kb upstream of the published first exon of the CG003 gene. In the mouse, the exon is deleted in crsp homozygous animals. Nevertheless, expression of the mouse cg003 gene was detected by RT-PCR using primers from the nondeleted middle part of the gene and RNA isolated from the heart of homozygous mutants indicating that the mouse gene contains an alternative nondeleted promoter.

We were unable to isolate mouse homologues of the human transcripts (CG014, CG017, CG029, CG037) and a putative pseudogene of transcription elongation factor-1d (TEF-1D) reported by Couch et al. (Genomics 36:86-99 (1996)) in this region of the genome. Hybridization of the human sequences with mouse BACs from the crsp deletion interval produced negative results, indicating that these sequences were not present in the mouse contig.

To identify additional genes within the deleted interval, we have obtained a draft sequence of three BACs (301H20, 337K7, and 389F6) covering the crsp interval. Sequencing was performed in the Genome Center of the Albert Einstein College of Medicine as a part of the Mouse Genome Sequencing Network Project. The genomic sequences were analyzed using GRAIL, GENESCAN, and BLAST programs. A number of potential exons were identified based on the computer analyses. The BLAST search also revealed several ESTs derived from this region of the genome. RT-PCR analyses using primers derived from each potential exon allowed us to narrow down the list of candidate genes. Simultaneously, we employed a direct comparative analysis of the mouse sequence with homologous human genomic sequence to confirm an evolutionary conservation of the identified exons.

One of the candidate genes is predicted to encode a novel protein with homology to the G protein coupled receptors (Schoneberg, et al., Mol. Cell. EndocrinoL 151:181-193 (1999)). Using RACE-PCR and screening of a mouse testis cDNA library, a complete sequence of the coding region has been determined. The open reading frame consists of 2,211 nucleotides and encodes a protein of 737 amino acids. The gene, which has been named Great (G protein-coupled receptor affecting testis descent, GenBank AF346501) is located proximal to cg003 in the crsp critical region. It is completely deleted in the mutant homozygotes and composed of 18 exons. Corresponding exons were identified in the human genomic sequence and were found to be well conserved both at the nucleotide (82% homology) and deduced amino acid (82% identity) level.

Expression of Great was analyzed by RT-PCR, revealing a distinct pattern of expression in wild-type adult organs. Expression was detected in the embryonic gonads (13.5 dpc) and adult gonads of males and females, as well as in male gubernaculum. The gene is also expressed in brain. No expression was detected in the other organs analyzed.

### Example 2

### Cloning of Human Gene

1. Materials and Methods

Gene Targeting and Production of Mice with Great Mutation

We isolated a genomic lambda phage clone containing exons 12-16 of the Great gene from a 5-prime HPRT library. An insertional targeting vector was constructed by conversion of the lambda phage into a plasmid, gapping the genomic insert with NheI, followed by linearization of the vector with the same restriction enzyme. We electroporated targeting construct DNA into AB2.1 ES cells and selected recombinant clones with G418 as described (Zheng, et al., Nucleic Acids Res. 27:2354-236017 (1999)). Analysis of the DNA from ES clones was performed by PCR with K/o primers derived from the vector backbone and from the NheI deleted fragment: vector 53527, 5′-AGAAGAGCAGAATAGCAGT-3′ (SEQ ID NO:14) and insert-reverse, 5′-ACC GCTCAGGGTCGAACT-3′ (SEQ ID NO:15). During homologous integration, the gap in the targeting vector is repaired and the PCR produces a 2 kb fragment.

We injected three selected independent clones into C57BL/6J blastocysts and reimplanted these into pseudopregnant female mice using standard procedures. Chimeric males were bred to C57BL/6J females to produce mice heterozygous for targeted allele. Great^{ko}/+ heterozygous males were bred to crsp/crsp homozygous females to produce Great^{ko}/crsp di-heterozygotes. Presence of the Great^{ko} mutant allele was detected by PCR of the tail DNA. Intercrossing of the Great^{ko}/+ heterozygotes produced homozygous animals which have been identified by long range PCR (Roche Biochemical) with primers outside the genomic fragment used in the targeting vector: forward primer, 5′-CATGGTGGGTAACCGGCT-3′ (SEQ ID NO: 16) and reverse primer, 5′-GCAAT-CCAAAGCCTCTAGC-3′ (SEQ ID NO:17). PCR conditions were denaturation at 94° C. for 2 min, 10 cycles of 92° C. for 10 sec, 55° C. for 30 sec, and 72° C. for 15 min, followed by 25 cycles of 92° C. for 10 sec, 55° C. for 30 sec, and 72° C. for 15 min+20 sec/per cycle with a final extension at 72° C. for 5 min. Presence of the sixteen kilobase fragment indicates presence of the wild-type allele.

RT-PCR analysis of the Gene Expression

Total RNA from mouse and human tissues was extracted with the TRIzol reagent (Life Technologies, Rockville, Md.). First strand cDNA was synthesized using the oligo(dT) primer and RETROscript kit (Ambion, Austin Tex.). The following primers were used for analysis of Great expression: Receptor2F, 5′-AGCAGTATGGT GGCTCCTCTG-3′ (SEQ ID NO:18); Receptor9F, 5′-ATGGTGGGTAACCGGCTC GAG-3′ (SEQ ID NO:19); Receptor 17F, 5′-AAAACAGCCCTTCAGACTGC-3′ (SEQ ID NO:20); Receptor16R, 5′-TCCCATAAAAGTTGCCGAAA-3′ (SEQ ID NO:21); Receptor 18R, 5′-CCACGATCCAGGAAGTGATT-3′ (SEQ ID NO:22). Cycling conditions were denaturation at 94° C. for 1 min, followed by 35 cycles of 94° C. for 30 sec, 55° C. for 30 sec, and 72° C. for 90 sec, with a final extension at 72° C. for 5 min.

Northern Blot Analysis

Northern blots with 2 μg of poly-A RNA isolated from embryos of different age were used (Clontech Labs, Palo Alto, Calif.). 1.3 kb 3′end cDNA of the GREAT gene was labeled by random priming (Stratagene, La Jolla, Calif.) and hybridized overnight in PerfectHyb Plus hybridization buffer (Sigma, USA) at 65° C. Blots were washed at 68° C. three times for 30 min with 0.1×SSC: 0.1% SDS and exposed to Kodak X-Omat film with one intensifying screen at −80° C. overnight.

Histopathological Analysis

Dissected testes were fixed in Bouin's solution (Sigma), embedded in paraffin, sectioned and stained with hematoxylineosin according to standard protocols. Photomicrographs were taken with an Axiolab microscope equipped with an automatic camera (Karl Zeiss, Germany).

Cloning of the Human GREAT Gene

Eighteen exons of the human GREAT gene were identified through the BLAST analysis of the human genomic DNA with the mouse CDNA sequence. Primers flanking both sides of the predicted ORF were designed: 5′UTR, 5′-TCAATTGCTGTAAACCTATGATTG-3′ (SEQ ID NO:23) and 3′UTR, 5′-CTTGCCGTTGGTAAAGATGAA-3′ (SEQ ID NO:24). These primers were used to amplify a cDNA with 2265 bp GREAT ORF. Both strands of the fragment were sequenced to confirm predicted splice sites/exons of the gene.

II. Results and Discussion

A. Genetic Targeting of the GREAT Gene in Mice Results in High Intraabdominal Cryptorchidism

To assess the role of the Great gene in the cryptorchid phenotype of the crsp mutation, we have produced animals with a mutant allele of the gene. We have targeted Great in ES cells using insertional type constructs (Zimmermann, et al., Mol. Reprod. Dev. 47:30-38 (1997)). Targeting of the gene resulted in the duplication of the exons 12-16 and insertion of the 10 kb vector DNA into the 16th intron. Heterozygous Great^{ko}/+ males and females showed a normal phenotype. Crosses of the heterozygous carriers with the original crsp mice produced crsp/Great^{ko} double heterozygotes. Intercrossing between heterozygous Great^{ko}/+ animals resulted in the production of Great^{ko}/Great^{ko} homozygotes. Analysis of the testicular phenotype in 14 Great^{ko}/Great^{ko} males and more than 50 crsp/Great^{ko} males reveals that these animals exhibit the same high intraabdominal cryptorchidism observed in the original crsp mutation. All tested heterozygous males had a fertile wild-type phenotype with a normal scrotum position of the testes. The size of the adult mutant testes is significantly decreased. At 60 days the average testis weight of the wild-type and heterozygous Great^{ko}/+ animals was 84.5±5.3 mg (n=8) and crsp/Great^{ko} testis was 31.0±1.1 (n=6) (63% reduction). Furthermore, histological examination reveals progressive degeneration of the spermatocytes, with absence of spermatids, and mature sperm. As in original crsp mutants, gubemacular development in Great knockout males is distinctively altered. The mutant mice have an extended thread-like gubernaculum, and the inguinal canal fails to form. Other urogenital structures in the mutant males are normal, including the seminal vesicles, prostate, and external genitalia. Mutant males exhibit normal mounting and copulatory behaviour. Vaginal plugs are present in females after copulation with mutant males, indicating that the males produce an ejaculate.

Analysis of Great gene expression at the RNA level by RT-PCR revealed presence of the specific RNA transcript in the crsp/Great^{ko} mutant animals. We detected Great mRNA using a primer pair derived from exons 9 and 16 (before vector insertion) and primers from exons 17-18 (after vector insertion). RT-PCR with primers from exon 2 and 18 failed to amplify expected 2 kb cDNA fragment from crsp/Great^{ko} brain RNA, however, indicating an inclusion of the backbone of the targeting vector DNA into the cDNA transcript and the absence of the properly processed Great cDNA.

The first stage of testicular descent, the transabdominal stage occurs in mice between day 15.5-17.5 d.p.c. We analyzed expression of the Great gene by Northern blot hybridization in mouse embryos at day 7 to 18 d.p.c. Expression of the Great gene was detected as early as in 7 days post coitum embryos with a stable expression on 11 d.p.c. and thereafter. Four different bands have been detected at the Northern blot, apparently representing several alternatively spliced transcripts. In RNA isolated from mouse or human brain, the main transcript is about 4.4 kb, in testis it is about 1.6 kb.

B. Cloning of the Human GREAT Gene

The human GREAT cDNA was predicted through comparisons of the mouse cDNA with corresponding human genomic sequence from chromosome 13q12-13 upstream of BRCA2 region. Subsequently, full-length cDNA has been isolated by RT-PCR from human gubemaculum RNA sample (GenBank accession number, AF453828). Comparison of the human and mouse cDNA sequences revealed that the human open reading frame starts 51 bp 5-prime to the first mouse ATG codon. This leads to the additional 17 amino acids at the N-terminal end of the human GREAT product. Overall identity of the mouse and human GREAT genes is 82% at nucleotide and amino acid level. The human gene has the same exon-intron structure as its mouse counterpart. Phylogenetic analysis of the mouse and human GREAT receptors shows that they belong to the LGR group of receptors, with a highest homology to the LGR7 receptor.

### Example 3

### Mutations in Human Population

I. Materials and Methods

Patient and Control DNA Samples

A total of 61 cases of idiopathic bilateral or unilateral cryptorchidism were used for the study. Twenty cases of the European origin were obtained from the French clinics and 41 samples of mixed origin from the Urology Department of Baylor College of Medicine, Texas. A total of 193 men of known fertility and absence of a clinical history of cryptorchidism were used as a control. Among them 62 samples were from France, 100 samples from Germany, and 31 samples from USA.

DHPLC Mutation Analysis and Sequencing

Mutation analysis was performed on an automated WAVE Nucleic Acid Fragment Analysis System according to conditions recommended by manufacturer (Transgenomic, Omaha, Neb., USA). Using available genomic sequence information we have designed 18 pairs of primers for amplification of each exon and flanking intron sequence of the human GREAT gene (Table 1). PCR was performed in 25 μl volume using ampliTaq (Perkin Elmer, Branchbury, N.J., USA) and a 5 μl aliquot was analyzed on the agarose gel. Prior to DHPLC analysis 15 μl (containing 100-300 ng of DNA) of the experimental sample was mixed with 5 μl of the control amplicon. PCR products were then denatured at 95° C. for 5 min and gradually cooled down to room temperature at 10° C./min decrements. Gradient parameters were determined based on size and G/C content of the amplicons. DHPLC conditions for successful resolution of heteroduplex formation have been established using the DNA melt software of the WAVEMaker software (Transgenomic). The column temperature was calculated using software supplied with the WAVE system. The predicted melting profiles for six exons of the gene required runs at two temperatures, the remaining twelve exons were analyzed at one temperature (Table 1). Chromatograms of the elution profiles obtained from the experimental samples were compared with those of the controls. All samples showing deviation from the wild-type profile were subject to direct sequencing. PCR fragments were separated on an agarose gel and purified using Ultrafree-DA spin columns (Millipore, Bedford, Mass., USA). Both strands of the DNA fragment were sequenced by the dye terminator method on an automated 373 DNA sequencing machine using the same primers as for PCR.

Expression of GREAT in Mammalian Cells

Full-length wild-type GREAT cDNA was amplified by RT-PCR from human gubemaculum RNA with primers 5′UTR and 3′UTR. The resulting 2.3 kb cDNA was subcloned into the eukaryotic cell expression vector PCR3.1 (Invitrogen, San Diego, Calif.). Efficient targeting of receptor to the cell surface was provided by internal GREAT signal peptide encoded by the first 36 amino acids of CDNA sequence. The plasmids were purified using the Concert Midi-prep plasmid preparation kit (Life Technologies, Rockvill, Mass.). The sequence of the construct was verified by sequencing of the both strands using gene-specific and vector-derived primers.

To produce targeted mutations in the GREAT cDNA we used QuikChange Site-Directed Mutagenesis kit from Stratagene (La Jolla, Calif.). Resulted cDNA sequence was verified by sequencing of both DNA strands. One kilobase CDNA EcoRI/HindIII fragment from the wild-type plasmid was substituted with the same fragment from the mutagenized plasmid, junction sites were verified by sequencing. This final plasmid, containing mutant GREAT cDNA, was used for further experiments.

293T cells derived from human embryonic kidney (HEK) fibroblast were maintained in DMEM supplemented with 10% FBS, 1 mM glutamine, and antibiotic/antimycotic mixture (all from Life Technologies, Rockvill, Mass.). For the cAMP determination experiment cells were seeded into 24 well plate 1-2 days prior transfection. The cells were transfected at 80% of confluency with 500 ng/well of plasmid of interest and 25 ng/well of reporter plasmid (pAP-tag5, GeneHunter Corporation, Nashville, Tenn.) in OptiMEM without antibiotic using FuGENE 6 transfection reagent (Roche, Indianapolis, Ind.). In co-transfection experiments cells were transfected with 250 ng/well of each plasmid (wild-type plus vector, or wild-type plus mutant). After 24 hours the efficiency of transfection was estimated by the activity of secreted alkaline phosphatase in the media using pNPP as a substrate (Sigma). Cells were stimulated with 0-60 nM porcine relaxin in the presence of 0.25 mM IBMX (Sigma) for 30 min, then harvested by aspiration and centrifuged. Pellet was washed briefly with PBS and extracted with cAMP extraction buffer (Amersham Pharmacia Biotech) for 40 min. Aliquots of supernatant were used in enzyme immunoassay (Amersham Pharmacia Biotech) for the cAMP determination. cAMP concentrations in each well were measured in duplicate. All experiments were repeated three times using cells from independent transfections.

TABLE 1

Primer sequences, PCR annealing temperature, and
DHPLC melting temperature for 18 exons of the hu-
man GREAT gene.

| | | | |
|---|---|---|---|
| | | PCR | DHPLC |
| | | Ann. | Melt |
| Exon | | Temp | Temp |
| No. | Primer Sequence | (° C.) | (° C.) |

| | | | |
|---|---|---|---|
| 1 | (SEQ ID NO:25) | 52 | 58 |
| | F: CATCAGAACTCCTGCTGAGGT | | |
| | (SEQ ID NO:26) | | |
| | R: TTTTTGGGAATTGCACTCATT | | |
| 2 | (SEQ ID NO:27) | 55 | 61 |
| | F: GAAAATTAAATGGGACAACACG | | |
| | (SEQ ID NO:28) | | |
| | R: CATAAAGTGAGGCACACATGG | | |
| 3 | (SEQ ID NO:29) | 50 | 56 |
| | F: CAAATTGTTAAATTTTGTCAGATGG | | |
| | (SEQ ID NO:30) | | |
| | R: TCTGTGCATCACAAATCATGT | | |
| 4 | (SEQ ID NO:31) | 55 | 55,57 |
| | F: GGCTGAATGAGTTCATTTAACA | | |
| | (SEQ ID NO:32) | | |
| | R: GCACAAGCAACACTATTGGG | | |
| 5 | (SEQ ID NO:33) | 50 | 56 |
| | F: CAAGCATGTGGCTTCTAGGG | | |
| | (SEQ ID NO:34) | | |
| | R: AAAACATCTTACACCACCACGA | | |
| 6 | (SEQ ID NO:35) | 55 | 55,57 |
| | F: CCAACATGAGAATAGGACTTCG | | |
| | (SEQ ID NO:36) | | |
| | R: TCGAGCCTTGAAAAAGTACCA | | |
| 7 | (SEQ ID NO:37) | 50 | 56 |
| | F: CATAATCATCATCAGTGGCTCA | | |
| | (SEQ ID NO:38) | | |
| | R: TCAAATAGTGGAAACTACATTAGCA | | |
| 8 | (SEQ ID NO:39) | 55 | 56,58 |

| | | | |
|---|---|---|---|
| | +TL,F: GGGGAGGCAGGTTTTATTTC | | |
| | (SEQ ID NO:40) | | |
| | R: AAGCTAGTGCTAGATGTCATTGC | | |
| 9 | (SEQ ID NO:41) | 55 | 55,60 |
| | F: CTCCCTAATCTTTGCCATACA | | |
| | (SEQ ID NO:42) | | |
| | R: TGGCTAGTTTCCTTAATCAGG | | |
| 10 | (SEQ ID NO:43) | 58 | 57 |
| | F: GGAGGAAAGATAGTAACAACTGGAA | | |
| | (SEQ ID NO:44) | | |
| | R: CAGTGTCAGACCAAGGCTGT | | |
| 11 | (SEQ ID NO:45) | 55 | 58 |
| | F: ACTACAGCAGACGCAAACCC | | |
| | (SEQ ID NO:46) | | |
| | R: CTAGTGGCAAAGCAGTCTCAC | | |
| 12 | (SEQ ID NO:47) | 55 | 56.5 |
| | F: TGGCTGACTCATACGGC | | |
| | (SEQ ID NO:48) | | |
| | R: GATTGGATGACAGGTTCCTT | | |
| 13 | (SEQ ID NO:49) | 50 | 55,57 |
| | F: GGAACTATCACCTCACCTT | | |
| | (SEQ ID NO:50) | | |
| | R: GACTTCATACATGTCGAATG | | |
| 14 | (SEQ ID NO:51) | 50 | 57.5 |
| | F: AAAACGTTTCATCTCAACACCA | | |
| | (SEQ ID NO:52) | | |
| | R: TTGTTGCATAGAAACACATTGC | | |
| 15 | (SEQ ID NO:53) | 55 | 57 |
| | F: CCCGATAGGACTGCAACTGT | | |
| | (SEQ ID NO:54) | | |
| | R: GTGCCCAGGATGTATAATTCA | | |
| 16 | (SEQ ID NO:55) | 55 | 54,60 |
| | F: GACATTGGAAACTGATGACAT | | |
| | (SEQ ID NO:56) | | |
| | R: CACAGTCTTGACTATGTTATCT | | |
| 17 | (SEQ ID NO:57) | 52 | 58 |
| | F: GGGTAAGGGATACTCAACCT | | |
| | (SEQ ID NO:58) | | |
| | R: GTGTCCTAGACTCTGGCCTT | | |
| 18 | (SEQ ID NO:59) | 55 | 56.5 |
| | F: GCATTGACTGCAAAGTGTTATTT | | |
| | (SEQ ID NO:60) | | |
| | R: CAAAAGCTGTCCCCTGTTTT | | |

II. Results and Discussion

Cryptorchidism is a common human congenital abnormality with a multifactorial etiology that likely reflects the involvement of endocrine, environmental and hereditary factors. Eighteen exons of the GREAT gene have been identified and subjected to mutation analysis using a recently developed high-throughput DHPLC approach (Xiao, et al. Hum. Mutat. 17:439-474 (2001)) and direct sequencing. We performed a mutation screen of the human GREAT gene in 61 cases of idiopathic unilateral or bilateral cyrptorchidism.

Two silent mutations were observed in exon 12, in both cryptorchid (40 out of 61) and normal men. These are A/G transversions at nucleotide position 957 and at position 993 (position 1 is taken as the first A of the initiation codon). Three different haplotypes were detected: A957 and A993, A957 and G993, G957 and G993. In 4 cryptorchid men an A/G transversion was observed at nucleotide position 1810 in the 5^{th} transmembrane domain. This nucleic acid substitution is predicted to result in a conservative amino acid change of an isoleucine to valine residue (1604V). The same sequence variant was detected in two out of thirty controls. Thus, this sequence variant is most likely a polymorphic allele not associated with cryptorchid phenotype.

In one of the patients of European origin we identified a unique mutation in exon 8. The patient had bilateral cryptorchidism with the gonads located in the inguinal canal at the external ring. The mutation resulted in an A to C nucleotide change at position 664 and was in heterozygous condition. This nucleotide change causes a missense substitution T222P. Analysis of the 192 control samples (162 from the same geographical area as the mutant carrier) did not reveal a sequence variation in this position. Importantly, no other variations in 18 exons of the GREAT of this patient have been detected. Direct sequencing of the 350 bp fragment upstream of the ATG codon also did not reveal any variations.

It was shown recently that relaxin, a hormone important for the growth and remodeling of reproductive and other tissues during pregnancy is capable of activating the GREAT receptor through an adenosine 3′,5′-monophosphate (cAMP)-dependent pathway. To investigate significance of the amino acid substitution in the mutant receptor we have analyzed relaxin-induced signal transduction in cells transfected with a wild-type and a mutant cDNA. cAMP concentration was determined in the transfected cells treated with increasing amounts of relaxin. Treatment of cells transfected with a wild-type or mutant 1604V GREAT cDNA with relaxin resulted in dose-dependent increases of cAMP production. In contrast, cAMP levels in cells transfected with a mutant T222P GREAT showed little response in cAMP production.

We have also co-transfected wild-type receptor cDNA with either the T222P mutant or an empty vector DNA to study possible interaction between wild-type and mutant polypeptides. It was found that stimulation causes the same increase in cAMP production in both cases, indicating that the non-functional mutant receptor does not affect signaling properties of the wild-type protein co-expressed in cells.

All references cited herein are fully incorporated by reference. Having now fully described the invention, it will be understood by those of skill in the art that the invention may be performed within a wide and equivalent range of conditions, parameters, and the like, without affecting the spirit or scope of the invention or any embodiment thereof.

What is claimed is:

1. A substantially pure protein consisting essentially of the amino acid sequence of SEQ ID NO:1.

2. An antibody that binds with specificity to the protein of claim 1.

3. A substantially pure polynucleotide consisting of a nucleotide sequence encoding the protein of claim 1.

4. The substantially pure polynucleotide consisting essentially of the nucleotide sequence of SEQ ID NO:2.

5. A vector comprising the polynucleotide of claim 3.

6. The vector of claim 5, wherein said vector is designed for the expression of the human GREAT protein, said vector comprising a coding region consisting of a nucleotide sequence encoding the protein of claim 1, said coding region being operably linked to a promoter.

7. A host cell transformed with the vector of either claim 5 or claim 6.

8. A method of assaying a test compound for its ability to bind to the human GREAT receptor, comprising:
(a) incubating a source of said human GREAT receptor protein according to claim 1 with:
i) an Insl3 ligand;
ii) said test compound; and
(b) determining the extent to which the binding of said Insl3 to said GREAT receptor is displaced by said test compound.

9. The method of claim 8, wherein said source of human GREAT receptor is a cell recombinantly engineered to express the protein of claim 1.

10. An assay to determine whether a human subject carries a gene that may lead to a disease or condition associated with abnormal GREAT expression, said method comprising:
(a) analyzing the GREAT gene of said subject; and
(b) concluding that said subject carries a gene that may lead to a disease associated with abnormal GREAT expression if said gene has one or more mutations when compared with the sequence of SEQ ID NO:2 and said gene encodes a protein that differs in sequence from SEQ ID NO:1.

11. The assay of claim 10, wherein said disease or condition associated with abnormal GREAT expression is cryptorchidism.

12. The assay of either claim 10 or claim 11, wherein said assay comprises amplifying the region of the GREAT gene by PCR amplification and then analyzing the amplification product for mutations.

13. An assay to determine whether a human subject carries a gene that may lead to a disease or condition associated with abnormal GREAT expression comprising:
(a) analyzing the GREAT receptor protein in said subject; and
(b) concluding that said subject carries a gene that may lead to a disease or condition associated with abnormal GREAT expression if said GREAT receptor protein has one or more mutations when compared to the sequence of SEQ ID NO:1.

14. The assay of claim 13, wherein said a disease or condition associated with abnormal GREAT expression is cryptorchidism

15. A substantially pure protein consisting essentially of the amino acid sequence of SEQ ID NO:3.

16. An antibody that binds with specificity to the protein of claim 15.

17. A substantially pure polynucleotide consisting of a nucleotide sequence encoding the protein of claim 15.

18. The substantially pure polynucleotide of claim 17, wherein said polynucleotide has the sequence of SEQ ID NO:4.

19. A vector comprising the polynucleotide of claim 17.

20. The vector of claim 19, wherein said vector is designed for the expression of the mouse GREAT protein, said vector comprising a coding region consisting of a nucleotide sequence encoding the protein of claim 13, and wherein said coding region is operably linked to a promoter.

21. A host cell transformed with the vector of either claim 19 or claim 20.

22. A method of assaying a test compound for its ability to bind to the mouse GREAT receptor, comprising:
(a) incubating a source of said mouse GREAT protein according to claim 15 with:
i) an Ins13 ligand;
ii) said test compound; and
(b) determining the extent to which the binding of said Ins13 to said GREAT receptor is displaced by said test compound.

23. The method of claim 22, wherein said source of mouse GREAT receptor is a cell recombinantly engineered to express the protein of claim 15.

24. A transgenic mouse comprising a mutated gene encoding the GREAT receptor protein SEQ ID NO:3 wherein said mutation results in cryptorchidism in male mice homozygous for said mutation.

25. A method of determining whether a test compound can be used to prevent cryptorchidism, comprising:
(a) interbreeding male and female transgenic mice according to claim 24, wherein said male mice are heterozygous for said mutated gene;
(b) administering said test compound to pregnant female mice resulting from the interbreeding of step (a);
(c) determining the extent to which the male progeny that are homozygous for said mutated gene have descended testes:
(d) comparing the results obtained with those from male progeny produced in a similar manner but in the absence of said test compound.
